(19) United States
(12) Patent Application Publication
(10) Pub. No.: US 2024/0347175 A1
(43) Pub. Date: Oct. 17, 2024

(54) MEDICAL IMAGE PROCESSING METHOD, MEDICAL IMAGE PROCESSING APPARATUS, AND STORAGE MEDIUM

(71) Applicant: CANON MEDICAL SYSTEMS CORPORATION, Tochigi (JP)

(72) Inventors: Fuyue WANG, Beijing (CN); Bing HAN, Beijing (CN); Qiqi XU, Beijing (CN)

(73) Assignee: CANON MEDICAL SYSTEMS CORPORATION, Tochigi (JP)

(21) Appl. No.: 18/633,744

(22) Filed: Apr. 12, 2024

(30) Foreign Application Priority Data
Apr. 12, 2023 (CN) 202310390508.6
Jan. 19, 2024 (JP) 2024-006733

(51) Int. Cl. G16H 30/40; G06V 10/26; G06V 10/72; G06V 10/774; G06V 10/82

(52) U.S. Cl. CPC G16H 30/40; G06V 10/26; G06V 10/72; G06V 10/7753; G06V 10/82

(57) ABSTRACT

A medical image processing method according to an embodiment of the present disclosure includes: training a deep neural network by using labeled image data; obtaining a first augmented image by carrying out a weak data augmentation on unlabeled image data; performing a predicting process on the first augmented image by using the deep neural network and determining whether each of the pixels in the first augmented image is able to serve as a pseudo-label on the basis of prediction information of the pixel; obtaining a second augmented image by carrying out a strong data augmentation on the first augmented image; training the deep neural network by using the second augmented image and the pseudo-labels; and updating the deep neural network on the basis of training results of the labeled image data and the unlabeled image data and processing a medical image by using the updated deep neural network.

### CROSS-REFERENCE TO RELATED APPLICATIONS

This application is based upon and claims the benefit of priority from Chinese Patent Application No. 202310390508.6, filed on Apr. 12, 2023; and Japanese Patent Application No. 2024-006733, filed on Jan. 19, 2024, the entire contents of all of which are incorporated herein by reference.

### FIELD

Embodiments described herein relate generally to a medical image processing method, a medical image processing apparatus, and a storage medium.

### BACKGROUND

Presently, methods for medical image segmentation can primarily be divided into methods using supervised deep learning and methods based on a gradation value analysis.

According to a method using supervised deep learning, supervised training for image segmentation or the like is performed, by using a Deep Neural Network (DNN) and labeled medical image data. With the deep neural network trained in this manner, it is usually possible to achieve a high level of accuracy. However, the method using supervised deep learning requires using a large amount of labeled medical image data. Accordingly, for tasks such as segmentation of a medical anatomical structure or segmentation in units of organ functions, it is difficult to label a medical image at the level of pixels. Those tasks not only take time, but also the costs thereof can be high.

In contrast, according to a segmentation method based on a gradation value analysis, segmentation is performed on a specific site or organ by analyzing gradation values of a medical image and calculating a Hessian matrix. However, because segmentation methods based on a gradation value analysis usually require parameter tuning, a problem remains where versatility is low, while robustness of results is low.

Further, other methods for medical image segmentation include methods using semi-supervised deep learning. According to a method using semi-supervised deep learning, by using both labeled medical image data and a large amount of unlabeled medical image data at the time of training a deep neural network, it is possible to achieve a high level of accuracy even when the amount of the labeled medical image data is small. Generally speaking, semi-supervised deep learning includes principles of consistency regularization and pseudo-labels or the like and also includes a connecting method combining both of the principles of consistency regularization and pseudo-labels.

According to a primary concept of the principle of consistency regularization, with respect to mutually the same medical images being input, prediction results called “predicted masks” are expected to be the same even if the medical images undergo small perturbation (e.g., a process such as a data augmentation performed on the medical images). However, generally speaking, using the principle of consistency regularization requires designing a number of neural network structures and pretext tasks. This requirement makes versatility and stability of the network low, and in addition, impacts the level of accuracy of predictions made by the deep neural network.

According to a theory of the principle of pseudo-labels, to begin with, one basic model (e.g., a deep network model using a convolutional neural network) is trained by using a small amount of labeled data. Subsequently, pseudo-labels are obtained by performing a predicting process on a large amount of unlabeled data, so as to train the model together by using both a large amount of unlabeled data having the pseudo-labels attached thereto and the small amount of labeled data. After that, pseudo-labels are obtained by performing a predicting process again on the large amount of unlabeled data by using the new trained model. Subsequently, the model is trained again, so that convergence is finally achieved after cycles. However, according to the principle of pseudo-labels in the method using the semi-supervised deep learning, when such pseudo-labels are obtained by performing a predicting process on unlabeled data while simply using a model, quality of the pseudo-labels is usually not very high. Consequently, levels of accuracy of training results and prediction results of the deep network model are not very high.

With the method using the semi-supervised deep learning, there are problems to be solved such as how to enhance the levels of accuracy of the training results and the prediction results and how to improve the level of accuracy of the prediction results by using unlabeled data, especially when the amount of labeled data is small.

### BRIEF DESCRIPTION OF THE DRAWINGS

FIG. 1 is a block diagram illustrating a functional configuration of a medical image processing apparatus according to a first embodiment;

FIG. 2 is a block diagram illustrating a configuration of an attention setting function according to the first embodiment;

FIG. 3 is a flowchart illustrating a procedure in a process (a medical image processing method) performed by the medical image processing apparatus according to the first embodiment;

FIG. 4A is a drawing illustrating an example of labeled image data being input;

FIG. 4B is a drawing illustrating an example of a prediction result (a segmentation result) obtained by a deep neural network by performing a predicting process on the labeled image data;

FIG. 4C is a drawing illustrating an example of Ground Truth (GT) of the labeled image data;

FIG. 5A is a drawing illustrating an example of unlabeled image data being input;

FIG. 5B is a drawing illustrating probability map average values of the unlabeled image data;

FIG. 5C is a drawing illustrating an example of pseudo-labels of the unlabeled image data;

FIG. 6 is a block diagram illustrating a functional configuration of a medical image processing apparatus according to a second embodiment;

FIG. 7 is a block diagram illustrating a configuration of an attention setting function according to the second embodiment;

FIG. 8 is a flowchart illustrating a procedure in a process (a medical image processing method) performed by the medical image processing apparatus according to the second embodiment;

FIG. 9 is a block diagram illustrating a functional configuration of a medical image processing apparatus according to a third embodiment;

FIG. 10 is a flowchart illustrating a procedure in a process (a medical image processing method) performed by the medical image processing apparatus according to the third embodiment;

FIG. 11A is a drawing illustrating an example of unlabeled image data input to a region of interest extracting function;

FIG. 11B is a drawing illustrating a prediction result obtained by a deep neural network with respect to the unlabeled image data; and

FIG. 11C is a drawing illustrating an example of region of interest data being extracted.

### DETAILED DESCRIPTION

A medical image processing method according to an embodiment of the present disclosure includes: training a deep neural network used for performing medical image processing, by using labeled image data being input; obtaining a first augmented image by carrying out a weak data augmentation on unlabeled image data being input; performing a predicting process on the first augmented image by using the deep neural network and determining whether or not each of the pixels in the first augmented image is able to serve as a pseudo-label on the basis of prediction information of the pixel; obtaining a second augmented image by carrying out a strong data augmentation on the first augmented image; training the deep neural network by using the second augmented image and the determined pseudo-labels; and updating the deep neural network on the basis of training results of the labeled image data and the unlabeled image data and further processing a medical image being input, by using the updated deep neural network.

Exemplary embodiments of a medical image processing method, a medical image processing apparatus, a storage medium, and a program will be explained in detail below, with reference to the accompanying drawings.

### First Embodiment

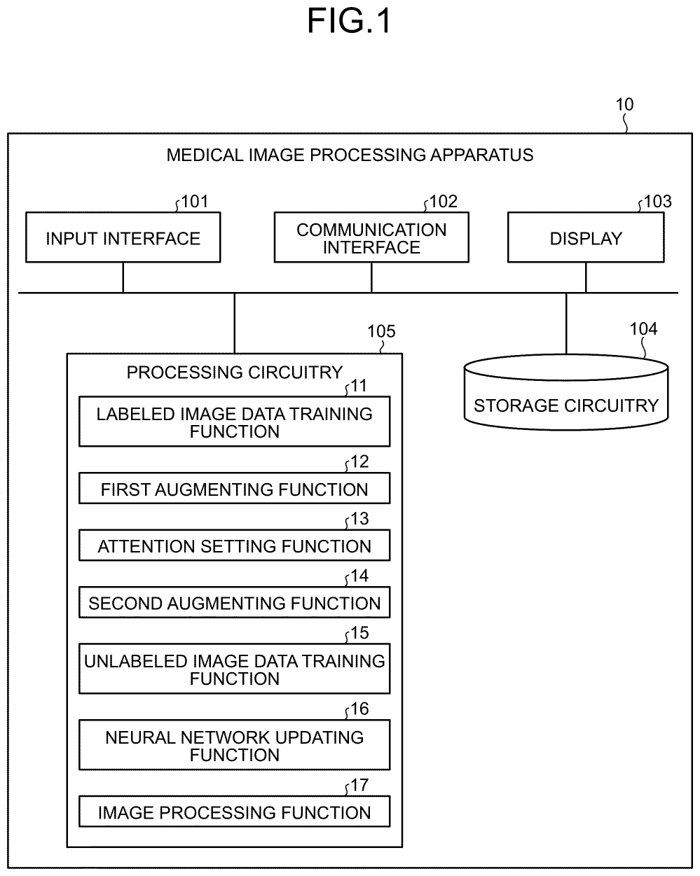

FIG. 1 is a block diagram illustrating a functional configuration of a medical image processing apparatus 10 according to a first embodiment. As illustrated in FIG. 1, the medical image processing apparatus 10 includes an input interface 101, a communication interface 102, a display 103, storage circuitry 104, and processing circuitry 105.

The input interface 101 is realized by using a trackball, a switch button, a mouse, a keyboard, a touchpad on which input operations can be performed by touching an operation surface thereof, a touch screen in which a display screen and a touchpad are integrally formed, contactless input circuitry using an optical sensor, audio input circuitry, and/or the like that are used for establishing various settings or the like. The input interface 101 is connected to the processing circuitry 105 and is configured to convert input operations received from a user such as a medical doctor to electrical signals and to output the electrical signals to the processing circuitry 105. Although the input interface 101 is provided in the medical image processing apparatus 10 in FIG. 1, the input interface 101 may be provided on the outside thereof.

The communication interface 102 may be a Network Interface Card (NIC) or the like and is configured to communicate with other apparatuses. For example, the communication interface 102 is connected to the processing circuitry 105 and is configured to acquire medical images from an ultrasound diagnosis apparatus serving as an ultrasound system or modalities other than the ultrasound system such as an X-ray Computed Tomography (CT) apparatus and a Magnetic Resonance Imaging (MRI) apparatus and configured to output the medical images to the processing circuitry 105.

The display 103 is connected to the processing circuitry 105 and is configured to display various types of information and various types of images output from the processing circuitry 105. For example, the display 103 is realized by using a liquid crystal monitor, a Cathode Ray Tube (CRT) monitor, a touch panel, or the like. For example, the display 103 is configured to display a Graphical User Interface (GUI) used for receiving an instruction from the user, as well as various types of images, and various types of processing results obtained by the processing circuitry 105. Although the display 103 is provided in the medical image processing apparatus 10 in FIG. 1, the display 103 may be provided on the outside thereof.

The storage circuitry 104 is connected to the processing circuitry 105 and is configured to store various types of data therein. More specifically, the storage circuitry 104 is configured to store therein at least various types of medical images for an image registration purpose, a fusion image obtained as a result of the registration process, and the like. For example, the storage circuitry 104 is realized by using a semiconductor memory element such as a Random Access Memory (RAM) or a flash memory, or a hard disk, an optical disk, or the like. Further, the storage circuitry 104 is configured to store therein programs corresponding to processing functions executed by the processing circuitry 105. Although the storage circuitry 104 is provided in the medical image processing apparatus 10 in FIG. 1, the storage circuitry 104 may be provided on the outside thereof.

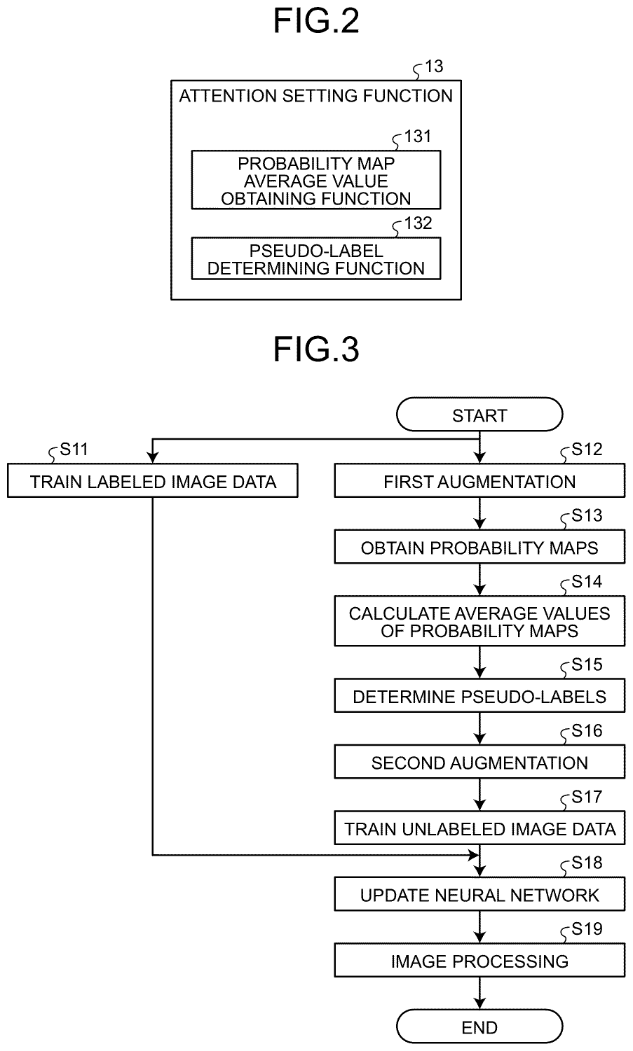

For example, the processing circuitry 105 is realized by using one or more processors. As illustrated in FIG. 1, the processing circuitry 105 includes a labeled image data training function 11, a first augmenting function 12, an attention setting function 13, a second augmenting function 14, an unlabeled image data training function 15, a neural network updating function 16, and an image processing function 17. FIG. 2 is a block diagram illustrating a configuration of the attention setting function 13 according to the first embodiment. As illustrated in FIG. 2, the attention setting function 13 includes a probability map average value obtaining function 131 and a pseudo-label determining function 132.

In the present example, the processing functions executed by the constituent elements of the processing circuitry 105 illustrated in FIG. 1 are recorded in the storage circuitry 104 of the medical image processing apparatus 10 in the form of computer-executable programs, for example. The processing circuitry 105 is realized with one or more processors configured to realize the processing functions corresponding to the programs, by reading and executing the programs from the storage circuitry 104. In other words, the processing circuitry 105 that has read the programs has the functions illustrated within the processing circuitry 105 in FIG. 1.

The term “processor” used in the above explanation denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)). When the processor is a CPU, for example, the one or more processors are configured to realize the functions by reading and executing the programs saved in the storage circuitry 104. In contrast, when the processor is an ASIC, for example, instead of having the programs saved in the storage circuitry 104, the programs are directly incorporated in the circuitry of the one or more processors. Further, the processors of the present embodiments do not each necessarily have to be structured as a single piece of circuitry. It is also acceptable to structure one processor by combining together a plurality of pieces of independent circuitry so as to realize the functions thereof. Furthermore, it is also acceptable to integrate two or more of the constituent elements in FIG. 1 into a processor, so as to realize the functions thereof.

Further, the functions of the processing circuitry 105 recorded in the storage circuitry 104 in the form of the computer-executable programs will be explained in detail later, with reference to the flowcharts.

Further, the storage circuitry 104 has stored therein a deep neural network (which may be called a “deep learning model”) used for performing image processing and training data used for training the deep neural network. The deep neural network in the present embodiment is trained by using a semi-supervised training scheme. The training data includes labeled image data having labels attached thereto and unlabeled image data having no labels attached thereto. The deep neural network may be an arbitrary type of neural network such as a Convolutional Neural Network (CNN) or a transformer, for example.

Further, in the present embodiment, by using the deep neural network, it is possible to perform, on a medical image being input, at least one selected from between segmentation of a medical anatomical structure and segmentation in units of organ functions. Examples of the segmentation of a medical anatomical structure include segmentation of the pancreas, segmentation of a lung lobe, and segmentation of the liver. Examples of the segmentation in units of organ functions include segmentation into hepatic segments. Each of the various types of segmentation processes corresponds to a prediction type of the deep neural network.

Further, in the present embodiment, a data augmentation is carried out to train the deep neural network. The data augmentation denotes a method by which data is artificially increased by applying a “transformation” to training-purpose image data (a medical image). There are various types of “transformations”. For example, the data augmentation has two types such as a “weak data augmentation” and a “strong data augmentation”. The weak data augmentation denotes, for example, performing, as a simple process on the medical image serving as image data, only a positional transformation such as a parallel displacement or an image mirroring process on the image, without changing the resolution or the contrast of the pixels and without increasing noise. The strong data augmentation denotes, for example, performing a great strain process on the medical image serving as image data, such as changing the sharpness (the resolution) or the contrast of the image, increasing Gaussian noise of the image, or randomly removing a partial region from the image.

FIG. 3 is a flowchart illustrating a procedure in a process (a medical image processing method) performed by the medical image processing apparatus 10 according to the first embodiment. In the following sections, the medical image processing method of the present embodiment will be explained in detail.

To begin with, at step S11 (a labeled image data training step), the labeled image data training function 11 trains the deep neural network, by using the labeled image data serving as training data and stored in the storage circuitry 104. The labeled image data training function 11 is an example of a “labeled image data training unit”.

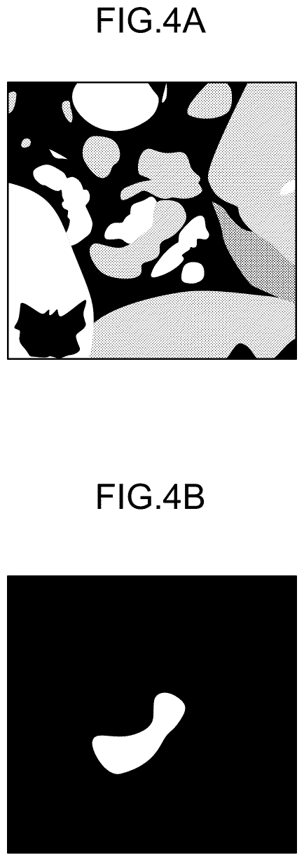

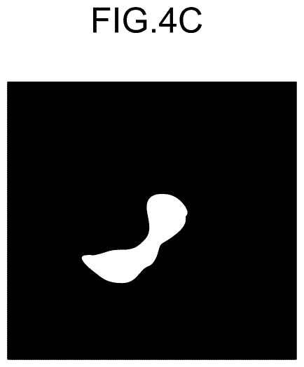

FIG. 4A is a drawing illustrating an example of the labeled image data being input. FIG. 4B is a drawing illustrating an example of a prediction result (a segmentation result) obtained by a deep neural network by performing a predicting process on the labeled image data. FIG. 4C is a drawing illustrating an example of Ground Truth (GT) of the labeled image data.

At the time of training the deep neural network by using the labeled image data, the labeled image data training function 11 performs the following processes:

For example, when one of the prediction types of the deep neural network is segmentation of the pancreas, the labeled image data training function 11 at first inputs a medical image of the abdomen of an examined subject (hereinafter, “patient”) as illustrated in FIG. 4A to the deep neural network, as the labeled image data. Subsequently, the labeled image data training function 11 performs a predicting process on the labeled image data on the basis of the deep neural network and further outputs a prediction result illustrated in FIG. 4B, for example, as a prediction result of the predicting process performed on the labeled image data. After that, on the basis of the difference between the prediction result (FIG. 4B) from the predicting process performed on the labeled image data and the GT (FIG. 4C) of the labeled image data, the labeled image data training function 11 obtains a loss function L₁ of the labeled image data as a training result. In this situation, the loss function L₁ is an example of the “training result of the labeled image data”.

An example of the loss function L₁ is presented in the expression below. In the expression below, “CE loss” denotes a cross entropy loss, whereas “Dice loss” denotes a Dice loss. N denotes the number of the pixels in the medical image; M denotes the number of the prediction types; P_{i,c} denotes a probability that a pixel i will be predicted as a prediction type c; and y1_{i,c} denotes a true label (GT) indicating that the pixel i is the prediction type c.

$${L}_{1} = CE loss + Dice loss \qquad ( Formula 1 )$$

$$CE loss = - {\sum }_{i = 1}^{N} {\sum }_{C = 1}^{M} y {1}_{i , c} log ( {P}_{i , c} ) \qquad ( Formula 2 )$$

$$Dice loss = 1 - \frac{{\sum }_{i = 1}^{N} {\sum }_{c = 1}^{M} y {1}_{i , c} {P}_{i , c}}{{\sum }_{i = 1}^{N} {\sum }_{c = 1}^{M} ( y {1}_{i , c} + {P}_{i , c} )} \qquad ( Formula 3 )$$

Further, at step S12 (a first augmenting step), the first augmenting function 12 randomly selects, with respect to each training session, an arbitrary piece of unlabeled image data as input data, from among all the pieces of unlabeled image data serving as the training data and stored in the storage circuitry 104. After that, the first augmenting function 12 obtains a first augmented image, by performing a weak data augmentation on the input unlabeled image data. The first augmenting function 12 is an example of a “first augmenting unit”.

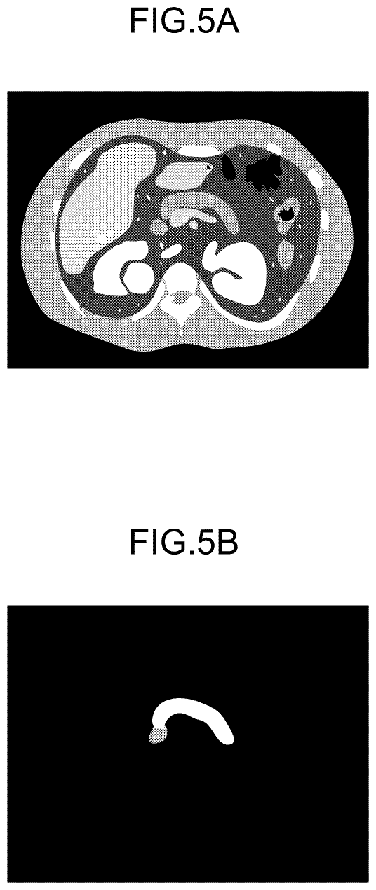

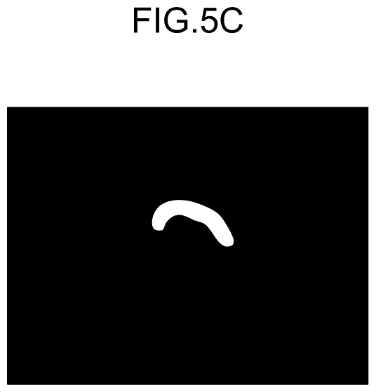

FIG. 5A is a drawing illustrating an example of unlabeled image data being input. As explained above, the weak data augmentation denotes, for example, performing, as a simple process on the medical image serving as the image data, only a positional transformation such as a parallel displacement or an image mirroring process on the image, without changing the resolution or the contrast of the pixels and without increasing noise. At step S12, for example, the first augmenting function 12 performs, on the medical image serving as the image data, a positional transformation to carry out a parallel displacement twice (a parallel displacement to the left and a parallel displacement to the bottom) and a positional transformation to carry out an image mirroring process once (left-right mirroring).

Subsequently, the attention setting function 13 performs the predicting process on the first augmented image by using the deep neural network and determines whether or not each of the pixels in the first augmented image is able to serve as a pseudo-label, on the basis of the prediction information of the pixel. The attention setting function 13 is an example of an “attention setting unit”.

To begin with, at step S13 (a probability map obtaining step), the attention setting function 13 obtains a probability map by performing the predicting process on the first augmented image, while using the deep neural network. More specifically, the attention setting function 13 obtains one or more probability maps by performing the predicting process on the first augmented image resulting from the positional transformation performed one or more times while using the deep neural network. For example, by using the deep neural network, the attention setting function 13 performs a predicting process on three first augmented images resulting from the positional transformations with the two times of the parallel displacement and the positional transformation with the one time of the image mirroring process and thus obtains three probability maps respectively corresponding to the three first augmented images.

Subsequently, at step S14 (a probability map average value calculating step), the attention setting function 13 calculates probability map average values of the first augmented image. The attention setting function 13 performs, on each of the one or more probability maps, a reverse positional transformation which is the reversal of the positional transformation performed one or more times at step S12 and further calculates a probability map average value of each of the pixels in the unlabeled image data, by using one or more probability maps resulting from the reverse positional transformation. The probability map average values are an example of the “prediction information of each of the pixels in the first augmented image”.

FIG. 5B is a drawing illustrating the probability map average values of the unlabeled image data. The probability expresses a degree of certainty by using a numerical value between “0” and “1”. When the degree of certainty is high, the numerical value is closer to “1”, and when the degree of certainty is low, the numerical value is closer to “0”.

In the example in FIG. 5B, the region in black in the drawing is a region where the probability map average values are “0”. When one of the prediction types of the deep neural network is segmentation of the pancreas, the probability map average values being “0” in the black region indicates that the probability that the region will be predicted as the pancreas is “0”. The region in white in the drawing is a region where the probability map average values are “1”. When one of the prediction types of the deep neural network is segmentation of the pancreas, the probability map average values being “1” in the white region indicates that the probability that the region will be predicted as the pancreas is “1”. Further, at the bottom left of the white region in the drawing, there is a gray region having ununiform gradation. The gray region is a region where the probability map average values are larger than “0”, but smaller than “1”. It is indicated that the probability that the grey region will be predicted as the pancreas is expressed with numerical values between “0” and “1”.

In this situation, step S13 (the probability map obtaining step) and step S14 (the probability map average value calculating step) are examples of the “probability map average value obtaining step”.

Next, at step S15 (a pseudo-label determining step), the attention setting function 13 judges, with respect to each of the pixels in the first augmented image, whether or not the probability map average value corresponding to the pixel is larger than a prescribed threshold value. Further, the attention setting function 13 determines the probability map average values of certain pixels larger than the prescribed threshold value to be pseudo-labels. Generally speaking, the prescribed threshold value is set in accordance with a distribution of probability map gradation values.

For example, when one of the prediction types of the deep neural network is segmentation of the pancreas, it is possible to use “0.5” as the prescribed threshold value for the probability map average values, in accordance with a specific distribution status of the gradation values. In that situation, the attention setting function 13 is configured to judge, with respect to each of the pixels, whether or not the probability map average value is larger than the prescribed threshold value “0.5”. Further, the attention setting function 13 determines the probability map average value of a pixel as a pseudo-label when the probability map average value is larger than the prescribed threshold value “0.5” and does not determine the probability map average value of a pixel as a pseudo-label when the probability map average value is equal to or smaller than the prescribed threshold value “0.5”. In the present embodiment, it is assumed that the probability map average values of certain pixels that are equal to or smaller than the prescribed threshold value “0.5” will not be used in the subsequent training of the deep neural network.

FIG. 5C is a drawing illustrating an example of pseudo-labels of the unlabeled image data. The pixels in the white region in FIG. 5C are the pixels determined as the pseudo-labels.

In this situation, step S13 (the probability map obtaining step), step S14 (the probability map average value calculating step), and step S15 (the pseudo-label determining step) are examples of the “attention setting step”.

Subsequently, at step S16 (a second augmenting step), the second augmenting function 14 obtains a second augmented image, by carrying out a strong data augmentation on the first augmented image obtained at step S12. As explained above, the strong data augmentation denotes, for example, performing a great strain process on the medical image serving as the image data, such as changing the sharpness (the resolution) or the contrast of the image, increasing Gaussian noise of the image, or randomly removing a partial region from the image. The second augmenting function 14 is an example of a “second augmenting unit”.

In this situation, step S16 does not necessarily need to be performed after step S15 and may be performed after step S12, for example.

After that, at step S17 (an unlabeled image data training step), the unlabeled image data training function 15 trains the deep neural network by using the second augmented image obtained at step S16 and the pseudo-labels determined at step S15. The unlabeled image data training function 15 is an example of an “unlabeled image data training unit”.

At the time of training the deep neural network by using the unlabeled image data, the unlabeled image data training function 15 performs the following processes:

For example, when one of the prediction types of the deep neural network is segmentation of the pancreas, the unlabeled image data training function 15 at first inputs the second augmented image obtained at step S16 to the deep neural network. Subsequently, the unlabeled image data training function 15 predicts a probability map of the second augmented image on the basis of the deep neural network and further outputs a prediction result (not illustrated). After that, on the basis of the difference between the prediction result (a predicted mask) obtained by predicting the probability map of the second augmented image and the pseudo-labels (FIG. 5C) obtained at step S15, the unlabeled image data training function 15 obtains a loss function Le of the unlabeled image data as a training result. In this situation, the loss function Le is an example of the “training result of the unlabeled image data”.

An example of the loss function Le is presented in the expression below. In the expression below, “CE loss” denotes a cross entropy loss, whereas “Dice loss” denotes a Dice loss. N denotes the number of the pixels in the medical image; M denotes the number of the prediction types; P_{i,c} denotes a probability that a pixel i will be predicted as the prediction type c; and y2_{i,c} denotes a pseudo-label (pseudo-GT) indicating that the pixel i is the prediction type c.

$${L}_{2} = CE loss + Dice loss \qquad ( Formula 4 )$$

$$CE loss = - {\sum }_{i = 1}^{N} {\sum }_{C = 1}^{M} y {2}_{i , c} log ( {P}_{i , c} ) \qquad ( Formula 5 )$$

$$Dice loss = 1 - \frac{{\sum }_{i = 1}^{N} {\sum }_{c = 1}^{M} y {2}_{i , c} {P}_{i , c}}{{\sum }_{i = 1}^{N} {\sum }_{c = 1}^{M} ( y {2}_{i , c} + {P}_{i , c} )} \qquad ( Formula 6 )$$

Subsequently, at step S18 (a neural network updating step), the neural network updating function 16 updates the deep neural network on the basis of the training result (the loss function L₁) of the labeled image data and the training result (the loss function L₂) of the unlabeled image data.

Further, steps S11 through S18 described above indicates the process of training the deep neural network on the basis of semi-supervised training. Although not illustrated, the training process usually needs to be repeated performed multiple times (tens to hundreds of times).

When the training has been performed a prescribed number of times, at step S19 (an image processing step), by using the deep neural network updated at step S18, the image processing function 17 processes a medical image that is subject to a predicting process and has been input to the deep neural network. The image processing function 17 is configured to process the input medical image, by using the deep neural network updated on the basis of the loss function L₁ and the loss function L₂.

More specifically, by using the deep neural network, the image processing function 17 is configured to perform at least one selected from between the segmentation of a medical anatomical structure and the segmentation in units of organ functions, on the input medical image. For example, when one of the prediction types of the deep neural network is segmentation of the pancreas, the image processing function 17 is configured to predict the segmentation of the pancreas with respect to a medical image of the abdomen of a patient input to the deep neural network, on the basis of the updated deep neural network and to further output a result of the segmentation of the pancreas. The image processing function 17 is an example of an “image processing unit”.

As explained above, in the medical image processing apparatus 10 according to the first embodiment, the labeled image data training function 11 is configured, at first, to train the deep neural network used for performing medical image processing, by using the labeled image data being input. Subsequently, the first augmenting function 12 is configured to obtain the first augmented image by carrying out the weak data augmentation on the input unlabeled image data. After that, the attention setting function 13 is configured to perform the predicting process on the first augmented image by using the deep neural network and to determine whether or not each of the pixels in the first augmented image is able to serve as the pseudo-label, on the basis of the prediction information (the probability map average value) of the pixel. Further, the second augmenting function 14 is configured to obtain the second augmented image by carrying out the strong data augmentation on the first augmented image. Subsequently, the unlabeled image data training function 15 is configured to train the deep neural network, by using the second augmented image and the pseudo-labels determined by the attention setting function 13. The image processing function 17 is configured to process the input medical image by using the deep neural network updated on the basis of the training result (the loss function L₁) of the labeled image data and the training result (the loss function Le) of the unlabeled image data. With this configuration, the medical image processing apparatus 10 according to the first embodiment is able to enhance the level of accuracy of the medical image processing.

For example, in the process (the medical image processing method) performed by the medical image processing apparatus 10 according to the first embodiment, while only a part of the pixels of which the prediction information (the probability map average values) is accurate is able to serve as the pseudo-labels, the other pixels of which the prediction information is unsatisfactory are unable to serve as the pseudo-labels. With this configuration, in the present embodiment, the pseudo-labels are optimized on the pixel levels. Thus, it is possible to increase a ratio of contribution of the pixels having the accurate prediction information to the network optimization and to inhibit the pixels having the unsatisfactory prediction information from impacting the network optimization. As a result, in the present embodiment, the training result at the time of training the deep neural network by using the unlabeled image data is optimized. In addition, in the present embodiment, when the predicting process is performed on a medical image about the segmentation of a medical anatomical structure or in units of organ functions while using the deep neural network, it is possible to achieve a higher level of prediction accuracy in the medical image processing.

Further, in the process (the medical image processing method) performed by the medical image processing apparatus 10 according to the first embodiment, the scheme is adopted by which, at the time of obtaining the probability map average values, the probability map average values are calculated through the plurality of positional transformations. With this configuration, in the present embodiment, it is possible to obtain the probability map average values that are more accurate and more certain, as compared to the situation where no positional transformation is performed. In addition, at the time of determining the pseudo-labels on the basis of the probability map average values, it is possible enhance the level of accuracy of the pseudo-labels.

As explained above, by implementing the medical image processing method (steps S11 through S19) based on the semi-supervised training, the medical image processing apparatus 10 according to the first embodiment is able to greatly improve the quality of the medical image segmentation, even when the amount of the labeled image data is small.

### Second Embodiment

Next, a medical image processing apparatus 10A and a medical image processing method according to a second embodiment will be explained.

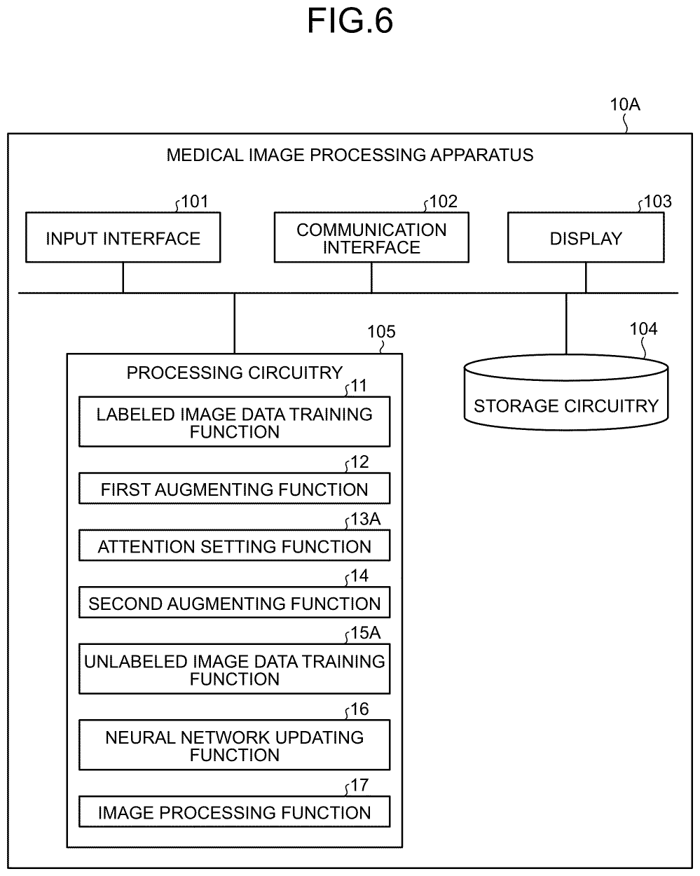

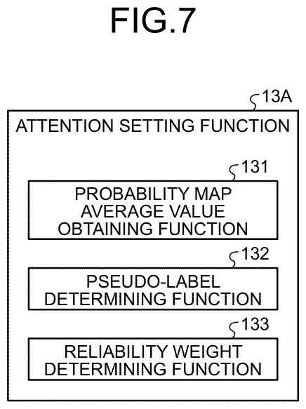

FIG. 6 is a block diagram illustrating a functional configuration of the medical image processing apparatus 10A according to the second embodiment. FIG. 7 is a block diagram illustrating a configuration of an attention setting function 13A according to the second embodiment.

As illustrated in FIG. 6, the processing circuitry 105 of the medical image processing apparatus 10A according to the second embodiment includes the labeled image data training function 11, the first augmenting function 12, an attention setting function 13A, the second augmenting function 14, an unlabeled image data training function 15A, the neural network updating function 16, and the image processing function 17. In other words, the processing circuitry 105 according to the second embodiment is different from that in the first embodiment for including the attention setting function 13A and the unlabeled image data training function 15A, in place of the attention setting function 13 and the unlabeled image data training function 15 of the processing circuitry 105 in the medical image processing apparatus 10 illustrated in FIG. 1.

As illustrated in FIG. 7, the attention setting function 13A according to the second embodiment includes the probability map average value obtaining function 131, the pseudo-label determining function 132, and a reliability weight determining function 133. In other words, the attention setting function 13A according to the second embodiment is different from the counterpart in the first embodiment for further including the reliability weight determining function 133, in addition to the probability map average value obtaining function 131 and the pseudo-label determining function 132 illustrated in FIG. 2.

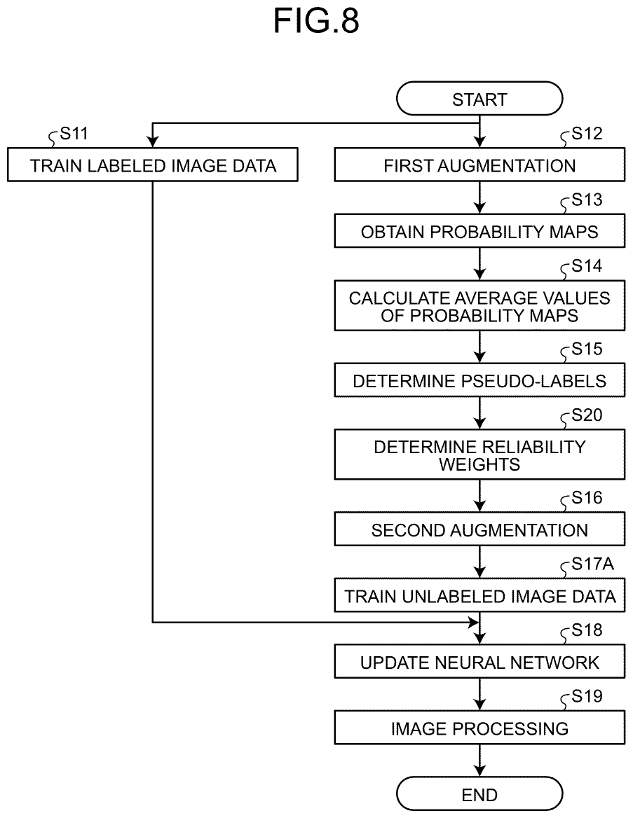

FIG. 8 is a flowchart illustrating a procedure in a process (a medical image processing method) performed by the medical image processing apparatus 10A according to the second embodiment. In the following sections, explanations that are the same as those in the first embodiment will be omitted, and only the differences will be explained.

As illustrated in FIG. 8, in the process (the medical image processing method) performed by the medical image processing apparatus 10A according to the second embodiment, step S20 (a reliability weight determining step) is added after step S15.

At step S20 (the reliability weight determining step), the reliability weight determining function 133 sets a reliability weight of each of the pixels in the first augmented image, in correspondence with the magnitude of the probability map average value of the pixel. The larger the probability map average value of the pixel is, the larger is the reliability weight to be determined. Conversely, the smaller the probability map average value of the pixel is, the smaller is the reliability weight to be determined. For example, for the probability map average values illustrated in FIG. 5B, for pixels of which the probability map average values are “1”, “0.9”, “0.6”, and “0”, the reliability weights of the pixels can be determined as “1”, “0.9”, “0.6”, and “0”, respectively.

Alternatively, it is also acceptable to adopt a scheme by which the reliability weights are binarized on the basis of the magnitudes of the probability map average values. For example, for certain pixels in the first augmented image of which the probability map average values are larger than “0.5”, the reliability weight of each of the pixels may be determined as “1”. For the other pixels in the first augmented image of which the probability map average values are equal to or smaller than “0.5”, the reliability weight of each of the pixels may be determined as “0”.

Instead of determining the reliability weight with respect to each of the pixels in the first augmented image, the reliability weight determining function 133 may be configured to set the reliability weights only for those pixels that were determined as the pseudo-labels at step S15. The reason is that the other pixels that were not determined as the pseudo-labels will not impact the training results in the subsequent training.

Further, as illustrated in FIG. 8, in the process (the medical image processing method) performed by the medical image processing apparatus 10A according to the second embodiment, a process at step S17A (an unlabeled image data training step) is performed in place of step S17 in FIG. 3.

At step S17A (the unlabeled image data training step), the unlabeled image data training function 15A trains the deep neural network, by using the second augmented image obtained at step S16, the pseudo-labels obtained at step S15, and the reliability weights obtained at step S20.

At the time of training the deep neural network by using the unlabeled image data, the unlabeled image data training function 15A performs the following processes:

For example, when one of the prediction types of the deep neural network is segmentation of the pancreas, the unlabeled image data training function 15A at first inputs the second augmented image obtained at step S16 to the deep neural network. Subsequently, the unlabeled image data training function 15A predicts a probability map of the second augmented image on the basis of the deep neural network and further outputs a prediction result. After that, on the basis of the prediction result from predicting the probability map of the second augmented image, the pseudo-labels obtained at step S15, and the reliability weights of the pixels obtained at step S20, the unlabeled image data training function 15A obtains a loss function L₂′ of the unlabeled image data as a training result taking the reliability weights into consideration.

An example of the loss function L₂′ is presented in the expression below. In the expression below, “CE loss′” denotes a cross entropy loss taking the reliability weights into consideration, whereas “Dice loss” denotes a Dice loss. N denotes the number of the pixels in the medical image; M denotes the number of the prediction types; w_{i,c} denotes a reliability weight when a pixel i will be predicted as the prediction type c; P_{i,c} denotes a probability that the pixel i will be predicted as the prediction type c; and y2_{i,c} denotes a pseudo-label (pseudo-GT) indicating that the pixel i is the prediction type c.

$${L}_{2}^{′} = CE {loss}^{′} + Dice loss \qquad ( Formula 7 )$$

$$CE {loss}^{′} = - {\sum }_{i = 1}^{N} {\sum }_{C = 1}^{M} {w}_{i , c} y {2}_{i , c} log ( {P}_{i , c} ) \qquad ( Formula 8 )$$

$$Dice loss = 1 - \frac{{\sum }_{i = 1}^{N} {\sum }_{c = 1}^{M} y {2}_{i , c} {P}_{i , c}}{{\sum }_{i = 1}^{N} {\sum }_{c = 1}^{M} ( y {2}_{i , c} + {P}_{i , c} )} \qquad ( Formula 9 )$$

In the process (the medical image processing method) performed by the medical image processing apparatus 10A according to the second embodiment, because the reliability weights on the pixel levels are introduced to the training result of the unlabeled image data, higher reliability weights are applied to the certain pixels having accurate prediction information (the probability map average values). As a result, in the present embodiment, it is possible to further strengthen the ratio of contribution of the pixels having the accurate prediction information to the deep neural network optimization. It is therefore possible to further enhance the level of accuracy of the image processing performed by the deep neural network.

As explained above, by implementing the medical image processing method (steps S11 through S20) based on the semi-supervised training, the medical image processing apparatus 10A according to the second embodiment is able to greatly improve the quality of the medical image segmentation, even when the amount of the labeled image data is small.

### Third Embodiment

Next, a medical image processing apparatus 10B and a medical image processing method according to a third embodiment will be explained.

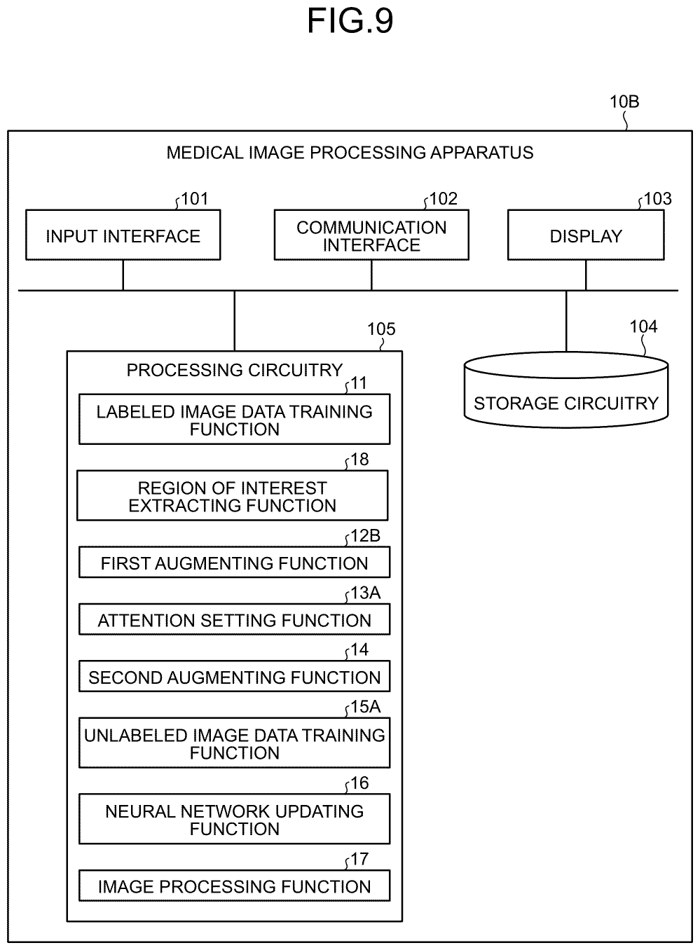

FIG. 9 is a block diagram illustrating a functional configuration of the medical image processing apparatus 10B according to the third embodiment.

As illustrated in FIG. 9, the processing circuitry 105 of the medical image processing apparatus 10B according to the third embodiment includes the labeled image data training function 11, a first augmenting function 12B, the attention setting function 13A, the second augmenting function 14, the unlabeled image data training function 15A, the neural network updating function 16, the image processing function 17, and a region of interest extracting function 18. In other words, the processing circuitry 105 according to the third embodiment is different from that in the second embodiment for further including the region of interest extracting function 18, in addition to the functions of the processing circuitry 105 in the medical image processing apparatus 10A illustrated in FIG. 6. Also, the processing circuitry 105 according to the third embodiment is different from that in the second embodiment for including the first augmenting function 12B, in place of the first augmenting function 12 of the processing circuitry 105 in the medical image processing apparatus 10A illustrated in FIG. 6.

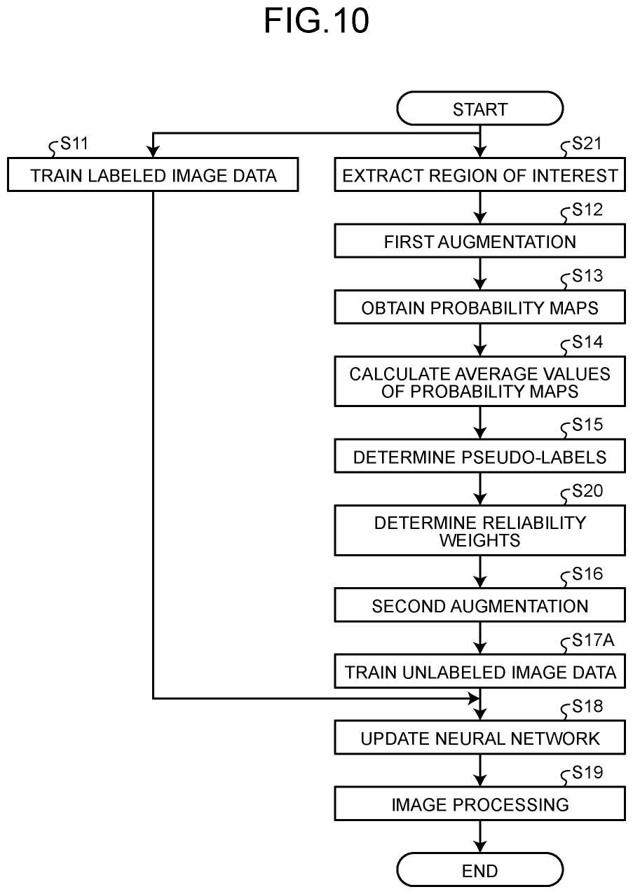

FIG. 10 is a flowchart illustrating a procedure in a process (a medical image processing method) performed by the medical image processing apparatus 10B according to the third embodiment. In the following sections, explanations that are the same as those in the first and the second embodiments will be omitted, and only the differences will be explained.

As illustrated in FIG. 10, in the process (the medical image processing method) performed by the medical image processing apparatus 10B according to the third embodiment, step S21 (a region of interest extracting step) is added before step S12, as compared to the second embodiment.

At step S21 (the region of interest extracting step), the region of interest extracting function 18 randomly selects, with respect to each training session, an arbitrary piece of unlabeled image data as input data, from among all the pieces of unlabeled image data serving as the training data and stored in the storage circuitry 104. After that, the region of interest extracting function 18 is configured to extract, with respect to the input unlabeled image data, a partial data including a region of interest in the unlabeled image data, as region of interest data, on the basis of a prediction result of the deep neural network.

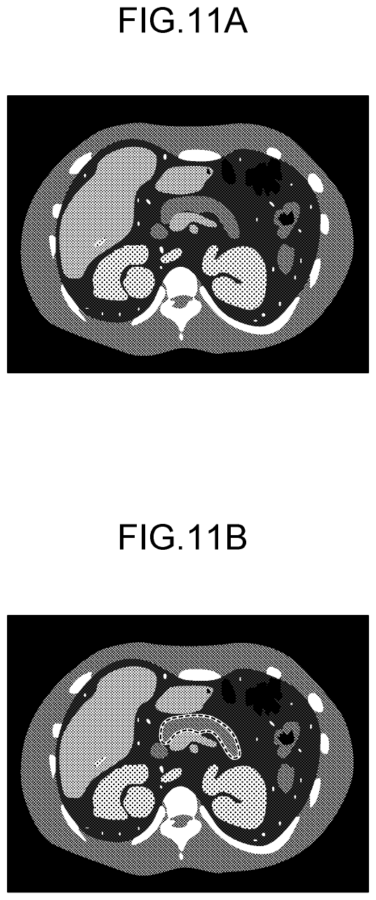

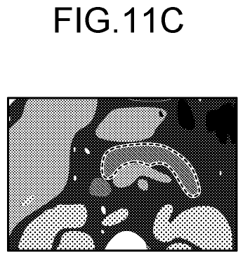

FIG. 11A is a drawing illustrating an example of the unlabeled image data input to the region of interest extracting function 18. FIG. 11B is a drawing illustrating a prediction result obtained by the deep neural network with respect to the unlabeled image data. In FIG. 11B, the region enclosed by the broken line is a segmentation result for a segmentation target (the pancreas). FIG. 11C is a drawing illustrating an example of the extracted region of interest data. It is possible to randomly select the region of interest, as long as the region of interest includes the entirety or a large part of the region of segmentation target (the pancreas) resulting from the predicting process.

Subsequently, at step S12 (the first augmenting step), the first augmenting function 12B obtains a first augmented image by carrying out a weak data augmentation on the extracted region of interest data.

In the process (the medical image processing method) performed by the medical image processing apparatus 10B according to the third embodiment, by extracting the region of interest data, it is possible to reduce the amount of the data to be used in the subsequent image processing and the subsequent training and to thus enhance efficiency of the training. In addition, in the present embodiment, the extracting process corresponds to eliminating a part of the data having a low ratio of contribution while making the percentage of the data having a high ratio of contribution relatively high. It is therefore possible to somewhat enhance the level of accuracy of the image processing performed by the deep neural network.

Further, the inventors performed a test to compare the process (the medical image processing method) performed by the medical image processing apparatus 10B according to the third embodiment with comparison examples such as a supervised technique and a semi-supervised technique. As a result, it was observed that the present embodiment was able to greatly improve the quality of the medical image segmentation, even when the amount of the labeled data was small.

For example, Table 1 presents a test result obtained when one of the prediction types of the deep neural network was segmentation of the pancreas. In the example of Table 1, Dice coefficients were used as an evaluation index for the test results. In the following sections, the evaluation index of the test results will be referred to as a “Dice index”.

In Table 1, “Comparison Example 1 (a supervised technique)” indicates a test result (a Dice index) of a supervised technique using only a small amount of labeled image data (the number of pieces in the training: 7). In Table 1, “Comparison Example 2 (a semi-supervised technique)” indicates a test result (a Dice index) of a semi-supervised technique using a small amount of labeled image data (the number of pieces in the training: 7) and unlabeled image data (the number of pieces in the training: 457). In Table 1, “Comparison Example 3 (a supervised technique)” indicates a test result (a Dice index) of a supervised technique using only labeled image data in a larger amount (the number of pieces in the training: 43) than in Comparison Example 1. In Table 1, “Third Embodiment (a semi-supervised technique)” indicates a test result (a Dice index) of a semi-supervised technique using a small amount of labeled image data (the number of pieces in the training: 7) and the unlabeled image data (the number of pieces in the training: 457). In this situation, the semi-supervised technique in Comparison Example 2 is different from the present embodiment for not including, at least, functions corresponding to the attention setting function 13, 13A, the unlabeled image data training function 15A, and the region of interest extracting function 18 of the present embodiment.

It is observed from Table 1 that the Dice index obtained by the present embodiment was higher than the supervised technique (Comparison Example 1) using only the small amount of labeled image data, was higher than the semi-supervised technique (Comparison Example 2), and further exceeded the training result of the supervised technique (Comparison Example 3) using only the larger amount of labeled image data. The Dice indices presented in Table 1 are customarily used for evaluating whether image segmentation algorithms for medical images are good or bad. It is indicated the larger the Dice value is, the better is the quality of the segmentation performed by the deep neural network.

TABLE 1

| | | Number of pieces | |
|---|---|---|---|
| | Number of pieces | of unlabeled | |
| | of labeled image | image data in | |
| Technique categories | data in training | training | Dice |
| Comparison example 1 | 7 | 0 | 0.647 |
| (Supervised technique) | | | |
| Comparison example 2 | 7 | 457 | 0.752 |
| (Semi-supervised | | | |
| technique) | | | |
| Comparison example 3 | 43 | 0 | 0.794 |
| (Supervised technique) | | | |
| Third embodiment | 7 | 457 | 0.798 |
| (Semi-supervised | | | |
| technique) | | | |

As explained above, by implementing the medical image processing method (steps S11 through S21) based on the semi-supervised training, the medical image processing apparatus 10B according to the third embodiment is able to greatly improve the quality of the medical image segmentation, even when the amount of the labeled image data is small.

### Other Embodiments

A number of embodiments have thus been explained; however, it is possible to carry out the present disclosure in various different modes other than those in the above embodiments.

For example, at the time of calculating the probability map average values, other schemes may be applied to step S13 (the probability map obtaining step) and step S14 (the probability map average value calculating step) in the first embodiment. For example, in a different scheme, at step S13 (the probability map obtaining step), the attention setting function 13 may be configured to perform the predicting process on the first augmented image by using each of a plurality of deep neural networks corresponding to training performed multiple times and to thus obtain a plurality of probability maps. Further, at step S14 (the probability map average value calculating step), the attention setting function 13 may be configured to calculate an average value of the plurality of probability maps, as probability map average values. According to the abovementioned different scheme, it is possible to obtain the probability map average values that are more accurate and more certain, as compared to the situation where the probability map average values from the plurality of probability maps are not calculated. In addition, at the time of determining the pseudo-labels on the basis of the probability map average values, it is possible to enhance the level of accuracy of the pseudo-labels.

Further, the constituent elements of the apparatuses illustrated in the drawings of the present embodiments are based on functional concepts. Thus, it is not necessarily required to physically configure the constituent elements as indicated in the drawings. In other words, specific modes of distribution and integration of the apparatuses are not limited to those illustrated in the drawings. It is acceptable to functionally or physically distribute or integrate all or a part of the apparatuses in any arbitrary units, depending on various loads and the status of use. Furthermore, all or an arbitrary part of the processing functions performed by the apparatuses may be realized by a CPU and a program analyzed and executed by the CPU or may be realized as hardware using wired logic.

Further, it is possible to realize the methods explained in the present embodiments, by causing a computer such as a personal computer or a workstation to execute a program prepared in advance. The program may be distributed via a network such as the Internet. Further, the program may be recorded on a non-transitory computer-readable recording medium such as a hard disk, a flexible disk (FD), a Compact Disk Read-Only Memory (CD-ROM), a Magneto Optical (MO) disk, a Digital Versatile Disk (DVD), or the like so as to be executed as being read by a computer from the recording medium.

According to at least one aspect of the embodiments described above, it is possible to enhance the level of accuracy of the medical image processing.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing method comprising:
a labeled image data training step of training a deep neural network used for performing medical image processing, by using labeled image data being input;
a first augmenting step of obtaining a first augmented image by carrying out a weak data augmentation on unlabeled image data being input;
an attention setting step of performing a predicting process on the first augmented image by using the deep neural network and determining whether or not each of pixels in the first augmented image is able to serve as a pseudo-label on a basis of prediction information of the pixel;
a second augmenting step of obtaining a second augmented image by carrying out a strong data augmentation on the first augmented image;
an unlabeled image data training step of training the deep neural network, by using the second augmented image and the pseudo-labels determined at the attention setting step; and
an image processing step of processing a medical image being input, by using the deep neural network updated on a basis of a training result of the labeled image data and a training result of the unlabeled image data.

2. The medical image processing method according to claim 1, wherein
the attention setting step includes:
a probability map average value obtaining step of obtaining probability maps by performing the predicting process on the first augmented image while using the deep neural network and calculating probability map average values of the first augmented image; and
a pseudo-label determining step of judging whether or not the probability map average value corresponding to each of the pixels in the first augmented image is larger than a prescribed threshold value and determining the probability map average values of certain pixels larger than the prescribed threshold value as the pseudo-labels.

3. The medical image processing method according to claim 2, wherein
the attention setting step further includes:
a reliability weight determining step of setting a reliability weight with respect to each of the pixels in the first augmented image, in correspondence with a magnitude of the probability map average value of the pixel; and
at the unlabeled image data training step, the deep neural network is trained by using the second augmented image, the pseudo-labels, and the reliability weights.

4. The medical image processing method according to claim 3, wherein
at the unlabeled image data training step,
the second augmented image is input to the deep neural network;
a probability map of the second augmented image is predicted on a basis of the deep neural network; and
a training result taking the reliability weights into consideration is obtained on a basis of the probability map of the second augmented image, the pseudo-labels, and the reliability weights of the pixels.

5. The medical image processing method according to claim 1, further comprising:
a region of interest extracting step at which, prior to the first augmenting step, partial data including a region of interest in the unlabeled image data is extracted as region of interest data, with respect to the input unlabeled image data, on a basis of a prediction result obtained by the deep neural network, wherein
at the first augmenting step, the first augmented image is obtained by carrying out a weak data augmentation on the region of interest data.

6. The medical image processing method according to claim 2, wherein the probability map average value obtaining step includes:
a probability map obtaining step of obtaining one or more probability maps by performing a predicting process on the first augmented image obtained through a positional transformation performed one or more times by using the deep neural network; and
a probability map average value calculating step of performing a reverse positional transformation which is a reversal of the positional transformation, on each of the one or more probability maps and further calculating a probability map average value of one or more probability maps resulting from the reverse positional transformation.

7. The medical image processing method according to claim 2, wherein the probability map average value obtaining step includes:
a probability map obtaining step of obtaining a plurality of probability maps, by performing the predicting process on the first augmented image while using each of two or more of the deep neural networks corresponding to the training performed multiple times; and
a probability map average value calculating step of calculating an average value of the plurality of probability maps as a probability map average value.

8. The medical image processing method according to claim 1, wherein, at the image processing step, at least one selected from between segmentation of a medical anatomical structure and segmentation in units of organ functions is performed on the medical image being input.

9. A medical image processing apparatus comprising processing circuitry configured:
to train a deep neural network used for performing medical image processing, by using labeled image data being input;
to obtain a first augmented image by carrying out a weak data augmentation on unlabeled image data being input;
to perform a predicting process on the first augmented image by using the deep neural network and to determine whether or not each of pixels in the first augmented image is able to serve as a pseudo-label on a basis of prediction information of the pixel;
to obtain a second augmented image by carrying out a strong data augmentation on the first augmented image;
to train the deep neural network by using the second augmented image and the determined pseudo-labels; and
to process a medical image being input, by using the deep neural network updated on a basis of a training result of the labeled image data and a training result of the unlabeled image data.

10. A non-transitory computer-readable storage medium storing therein a program that causes a computer to perform:
training a deep neural network used for performing medical image processing, by using labeled image data being input;
obtaining a first augmented image by carrying out a weak data augmentation on unlabeled image data being input;
performing a predicting process on the first augmented image by using the deep neural network and determining whether or not each of pixels in the first augmented image is able to serve as a pseudo-label on a basis of prediction information of the pixel;
obtaining a second augmented image by carrying out a strong data augmentation on the first augmented image;
training the deep neural network by using the second augmented image and the determined pseudo-labels; and
processing a medical image being input, by using the deep neural network updated on a basis of a training result of the labeled image data and a training result of the unlabeled image data.
